(19) United States
(12) Patent Application Publication
(10) Pub. No.: US 2015/0018714 A1
(43) Pub. Date: Jan. 15, 2015

(54) STEP-SHAPED ENDOMETRIAL CURETTE

(71) Applicant: Hong-Ming CHEN, New Taipei (TW)

(72) Inventor: Hong-Ming CHEN, New Taipei (TW)

(21) Appl. No.: 13/965,984

(22) Filed: Aug. 13, 2013

(30) Foreign Application Priority Data
Jul. 12, 2013 (TW) 102213258

(51) Int. Cl. A61B 10/02

(52) U.S. Cl. CPC A61B 10/0291
USPC 600/570

(57) ABSTRACT

A step-shaped endometrial curette is provided. The step-shaped endometrial curette includes a handle, a curetting bar, and a collection cup. The handle is connected to a first end portion of the curetting bar. A second end portion of the curetting bar has a distal end formed with a plurality of mushroom-shaped curetting plates for removing endometrial tissues by scraping. The collection cup is slidably connected between the first end portion and the mushroom-shaped curetting plates and serves to hold the removed endometrial tissues. As the mushroom-shaped curetting plates are arranged stepwise, and the curetting bar is freely flexible to adapt to the internal shape and angles of the uterus, specimens can be taken from inside the uterus without dilatation of the cervix or anesthesia. Besides, the collection cup can collect all the removed endometrial tissues to enable sufficient and thorough pathological examination.

### BACKGROUND OF THE INVENTION

1. Technical Field

The present invention relates to an endometrial curette and, more particularly, to a step-shaped endometrial curette.

2. Description of Related Art

Endometrial cancer, or corpus uteri cancer, is a malignant gynecologic tumor common to women in Europe and the United States. The incidence rate of endometrial cancer is higher than those of cervical cancer and ovarian cancer.

In Taiwan, cancers have been the leading causes of disease-related death for the last three decades, with lung cancer, liver cancer, and colorectal cancer invariantly being the top three causes, for men and women alike. Meanwhile, early detection and treatment of breast cancer and cervical cancer, two of the most common female cancers, have been achieved, thanks to effective public health education and the generally accepted practice of cancer screening. The death rate of cervical cancer, in particular, has dropped on an annual basis from the highest of all cancers of about thirty years ago to the ranking as No. 10. For that, the most credit goes to extensive application of the cervical cancer screening test, or better known as the “Pap smear test”, which is a pathological examination conducted on cells and tissues to enable early detection and treatment either.

However, the incidence rate of endometrial cancer in Taiwan has increased by 16% in recent years. Endometrial cancer used to have more or less the same incidence rate in Taiwan as breast cancer, but now the incidence rate of the former has increased significantly. One possible cause is that postmenopausal women in Taiwan have been more likely than before to consume exogenous estrogen without knowing it. Furthermore, as the living and eating habits in Taiwan are gradually westernized, more and more animal fat and sugar-containing food have been consumed. Risk factors such as obesity have also added to the incidence rate of endometrial cancer.

The diagnosis of endometrial cancer relies on the assistance of images taken by medical ultrasonography or computerized tomography. In addition, it is necessary to take biopsy specimens from the endometrium, using medical equipment inserted into the uterus through the vagina and the cervix, and the specimens taken are subsequently dyed and fixed for pathological diagnosis. Generally speaking, test results derived from endometrial cells tend to be with high false negative rate, as are those obtained by vaginal douching or smearing; hence, it is difficult to make an accurate diagnosis with endometrial cell specimens. By contrast, the accuracy of a diagnosis made by curettage and tissue biopsy can be as high as 90%. The only reliable approach to accurate diagnosing endometrial abnormalities, therefore, is to perform pathological examination on endometrial tissues.

During the diagnosis of endometrial cancer, it is common to find endometrial polyps or over thickening of the endometrium, both of which are benign conditions. Irregular or cystic hyperplasia of the endometrium, on the other hand, stands 50% or so risks of mutation and variation, which may result in endometrial dysplasia and consequently endometrial cancer.

If not treated in time, endometrial cancer cells may invade the muscle layer, or even penetrate the serosa of the uterus, and then spreading out through the lymphatic system or the vascular system. Should this occur, the patient's condition is bound to worsen and, if deteriorating or spreading out beyond early treatment, may become fatal. According to statistics, the incidence rate of endometrial cancer in Taiwan has risen by 16% in recent years, which is a warning not to be overlooked.

In diagnosing endometrial cancer, dilatation and curettage devices are typically used. Apart from dilating the cervical canal with a proper tool in advance, it is generally required that the patient be anesthetized. The dilating and curetting process is now briefly stated as follows. To begin with, the cervix is exposed after the insertion of a vagina speculum. Then, the uterus is fixed in proper position with a tenaculum. The cervical canal is subsequently dilated to a suitable and optimum diameter by dilators, allowing a curette to be inserted into the uterine cavity and curette the endometrium for pathological diagnosis. As the foregoing process is complicated and painful, endometrial curettage is not a very popular procedure among patients, and hesitation on the patients' side to receive the required procedure often delays diagnosis.

FIG. 1 shows a conventional endometrial curette 200, which is made of stainless steel and is therefore hard, inflexible, and prone to cause discomfort to patients. Moreover, as the amount of tissues that a stainless steel curette can remove by scraping is limited, repeated curettage is necessary, but the time required for sampling will be prolonged as a result.

FIG. 2 shows another conventional endometrial curette 300, which is an extraction and curettage tube made of plastic. Two concave grooves 301 are provided near the tip, and on two opposite sides, of the curette 300 for curetting and for extracting endometrial tissues. Nevertheless, the area of contact between the curette 300 and the endometrium is still rather small, requiring the curetting action to be performed over and over again; in consequence, endometrial tissue specimens cannot be rapidly and sufficiently taken.

Both endometrial curettes 200 and 300 cause considerable discomfort to patients and require anesthesia, which makes it impossible to carry out the curetting process in an outpatient setting; that is to say, an operating room must be scheduled for the process. According to the above, the conventional endometrial curettes leave much to be desired and have hindered the promotion of endometrial cancer screening.

### BRIEF SUMMARY OF THE INVENTION

The present invention provides a step-shaped endometrial curette whose curetting bar is freely flexible to adapt to the internal, shape and angles of the uterus; therefore, specimens can be taken without cervical dilatation or anesthesia and it is also safer in terms of to minimize the risks of uterine injury or even to penetrate the uterine body. In addition, the collection cup of the endometrial curette is so designed that it can be pushed upward and pressed tightly against the opening of the cervix to collect all the scraped-off endometrial tissues, thereby enabling for sufficient and thorough pathological examination.

The present invention provides a step-shaped endometrial curette, comprising: a handle; a curetting bar having a first end portion and a second end portion, wherein the first end portion is connected to the handle, and the second end portion has a distal end formed with a plurality of mushroom-shaped curetting plates; and a collection cup slidably connected between the first end portion and the mushroom-shaped curetting plates.

Implementation of the present invention produces the following advantageous effects:

1. The design of the collection cup allows endometrial tissues to be collected conveniently and completely;

2. Specimen collecting can be done without cervical dilatation or anesthesia;

and

3. The process of collecting endometrial tissues is simplified to increase patients' willingness to take the endometrial test.

The features and advantages of the present invention are detailed hereinafter with reference to the preferred embodiments. The detailed description is intended to enable a person skilled in the art to gain insight into the technical contents disclosed herein and implement the present invention accordingly. In particular, a person skilled in the art can easily understand the objects and advantages of the present invention by referring to thee disclosure of the specification, the claims, and the accompanying drawings.

### BRIEF DESCRIPTION OF THE SEVERAL VIEWS OF THE DRAWINGS

FIG. 1 shows a conventional endometrial curette;

FIG. 2 shows another conventional endometrial curette;

FIG. 3A is a perspective view showing the first aspect of the step-shaped endometrial curette in an embodiment of the present invention;

FIG. 3B is a perspective view showing the second aspect of the step-shaped endometrial curette in an embodiment of the present invention;

FIG. 4 is a sectional view of the mushroom-shaped curetting plates in an embodiment of the present invention;

FIG. 5 is a perspective view showing how a collection cup is slidably connected to a curetting bar according to an embodiment of the present invention;

FIG. 6A is a sectional view of the collection cup in an embodiment of the present invention;

FIG. 6B is a top view showing the first aspect of the collection cup in an embodiment of the present invention;

FIG. 6C is a top view showing the second aspect of the collection cup in an embodiment of the present invention;

FIG. 6D is a top view showing the third aspect of the collection cup in an embodiment of the present invention;

FIG. 7 is a perspective view showing how the collection cup is engaged with and stopped at an anti-slide projection according to an embodiment of the present invention;

FIG. 8 is a sectional view of the anti-slide projections in an embodiment of the present invention;

FIG. 9 shows how the step-shaped endometrial curette in an embodiment of the present invention is used;

FIG. 10 is a perspective view showing how the curetting bar can be broken off at a notch according to an embodiment of the present invention;

FIG. 11 is a perspective view showing how the collection cup in FIG. 10 is removed according to an embodiment of the present invention;

FIG. 12 is a perspective view showing how the curetting bar can be separated from the handle according to an embodiment of the present invention; and

FIG. 13 is a perspective view showing how the collection cup in FIG. 12 is removed according to an embodiment of the present invention.

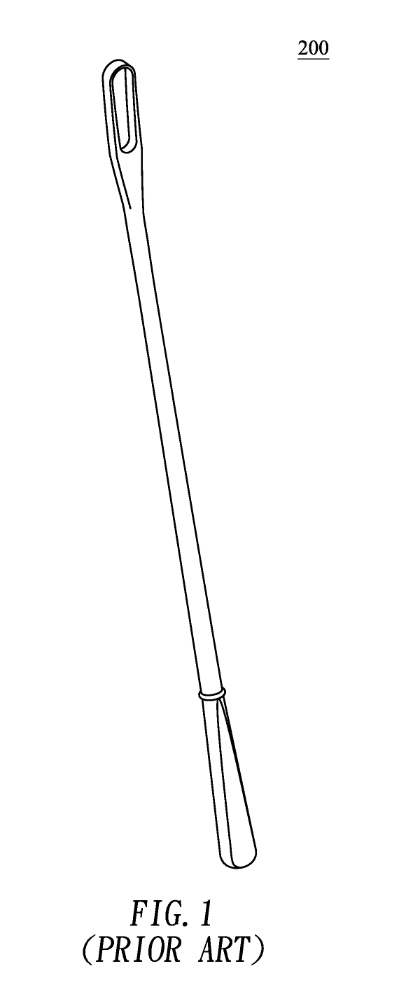

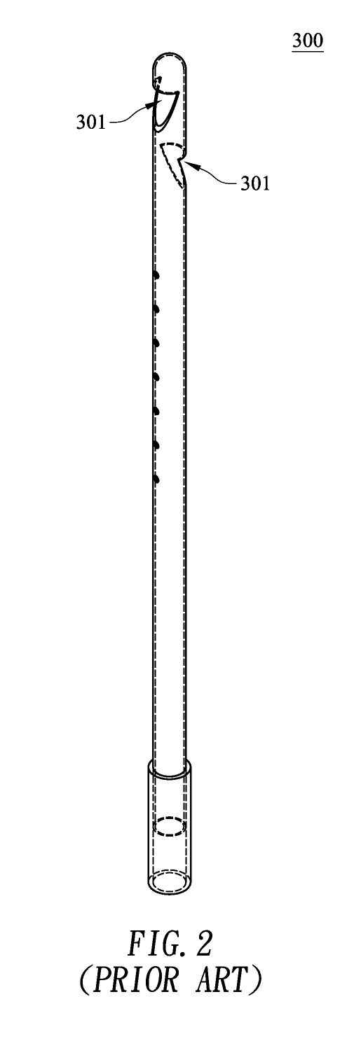

### DETAILED DESCRIPTION OF THE INVENTION

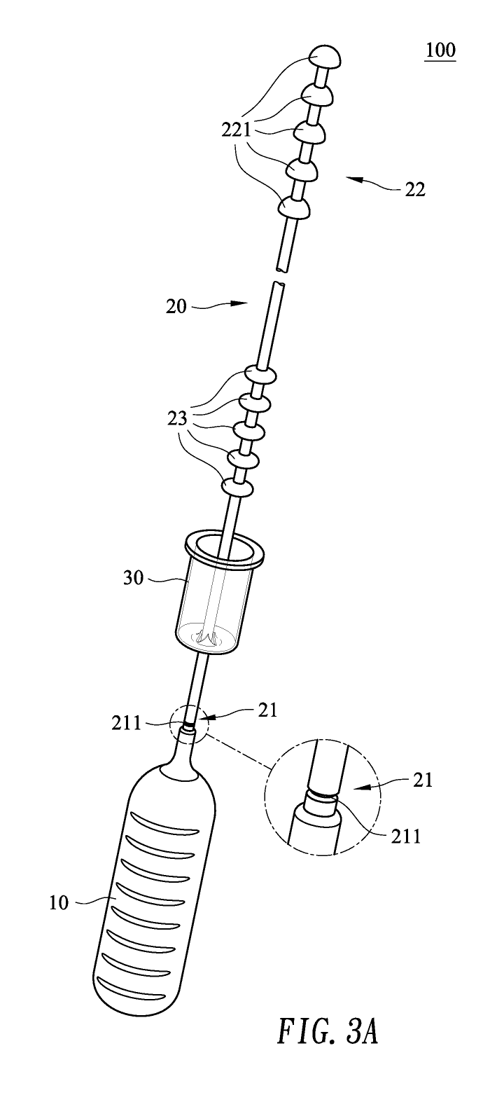

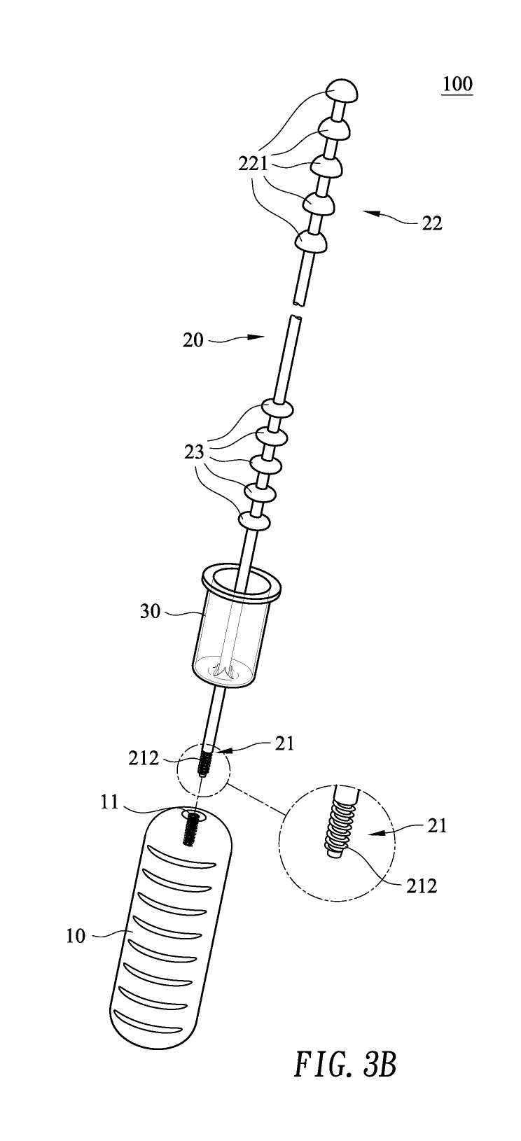

Referring to the embodiment shown in FIG. 3A and FIG. 3B, the step-shaped endometrial curette 100 includes a handle 10, a curetting bar 20, and a collection cup 30.

The handle 10 is configured to be held by the user to facilitate operation of the endometrial curette 100. The present invention imposes no limitations on the form of the handle 10, provided that the handle 10 can be held and operated with ease.

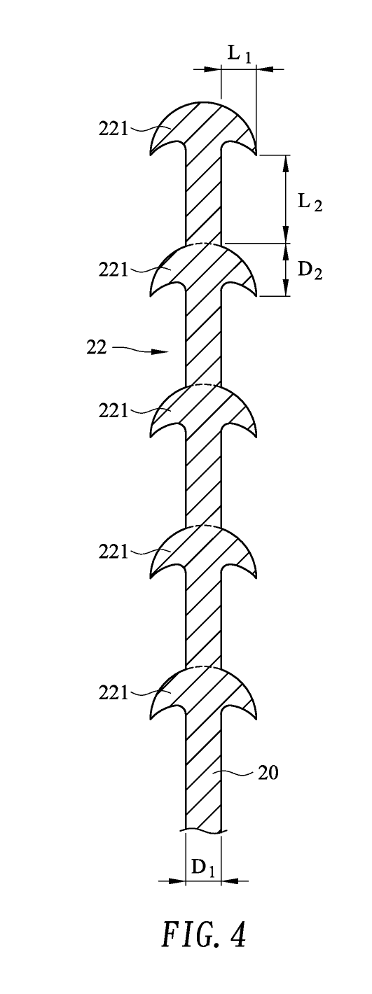

As shown in FIG. 3A and FIG. 4, the curetting bar 20 has a first end portion 21 and a second end portion 22. The first end portion 21 can be connected to the handle 10, with a notch 211 formed at the joint therebetween so that the curetting bar 20 can be broken off at the notch 211. The curetting bar 20 has a bar body whose cross-sectional diameter D₁ may be 0.2 cm.

Alternatively, the first end portion 21 also can be connected to the handle 10 by means of screwing. For example, as shown in FIG. 3B, the first end portion 21 can form with screw threads 212, and a connecting end of the handle 10 can form with a threaded hole 11. Thus, the handle 10 and the curetting bar 20 can be assembled by screwing the screw threads 212 into the threaded hole 11. It is also very easy to separate the handle 10 and the curetting bar 20. The curetting bar 20 should be thrown away after using and the used handle 10 is reusable and can be connected to a new curetting bar 20. Hence, the manufacturing cost of the handle 10 can be reduced and it is also environmentally friendly.

As shown in FIG. 4, the distal end of the second end portion 22 of the curetting bar 20 is formed with a plurality of mushroom-shaped curetting plates 221, each resembling a mushroom with a domed cap. For example, each mushroom-shaped curetting plate 221 may include a hemispheric body and a column, wherein the column is connected to the center of the bottom surface of the hemispheric body such that the hemispheric body is located at the top end of the column. The thickness of the hemispheric body is gradually reduced along the radial direction, i.e., from the center outward, thus forming a relatively sharp peripheral edge for curetting the endometrium easily.

The mushroom-shaped curetting plates 221 are perpendicular to the bar body of the curetting bar 20 and are formed at an equal interval along the distal end of the second end portion 22 in a step-like manner. More particularly, the projecting portions of the mushroom-shaped curetting plates 221 are parallel to one another and are perpendicular to the bar body of the curetting bar 20. The domed cap of each mushroom-shaped curetting plate 221 has a length L₁ extending outward from the bar body of the curetting bar 20. The length L₁ can also be viewed as the distance between the edge of each hemispheric body and the bar body of the curetting bar 20. The length L₁ ranges from 0.05 cm to 0.1 cm and may be either 0.05 cm or 0.1 cm, depending on the overall diameter of the endometrial curette 100.

The number of the mushroom-shaped curetting plates 221 may be five, and the interval L₂ at which the mushroom-shaped curetting plates 221 are arranged may be 0.5 cm, wherein the interval L₂ of the mushroom-shaped curetting plates 221 is defined as the distance between the top end of the hemispheric body of a mushroom-shaped curetting plate 221 and the bottom surface of the hemispheric body of the immediately above mushroom-shaped curetting plate 221. Besides, the greatest thickness D₂ of each mushroom-shaped curetting plate 221, i.e., the greatest thickness of the hemispheric body, may be 0.2 cm. Therefore, when there are five mushroom-shaped curetting plates 221, the distance between the bottom surface of the lowermost mushroom-shaped curetting plate 221 and the top end of the uppermost mushroom-shaped curetting plate 221 is 3 cm, which distance is ideal for moving the step-shaped endometrial curette 100 up and down to curette the endometrial contents. The dimensions of the mushroom-shaped curetting plates 221, however, are by no means limited to the foregoing. For the sake of safety, the uppermost mushroom-shaped curetting plate 221 has a smooth hemispheric surface to prevent the tip of the curette from injuring or piecing through the uterus during the curettage.

Moreover, as the step-shaped endometrial curette 100 can be provided with a plurality of mushroom-shaped curetting plates 221, each addition of a mushroom-shaped curetting plate 221 will increase the area of contact between the endometrial curette 100 and endometrial tissues. Further, the disk-like, mushroom-shaped curetting plates 221 can make contact with endometrial tissues in all directions (360 degree) to enable sufficient curettage of endometrial tissues and shorten the time required for specimen collecting.

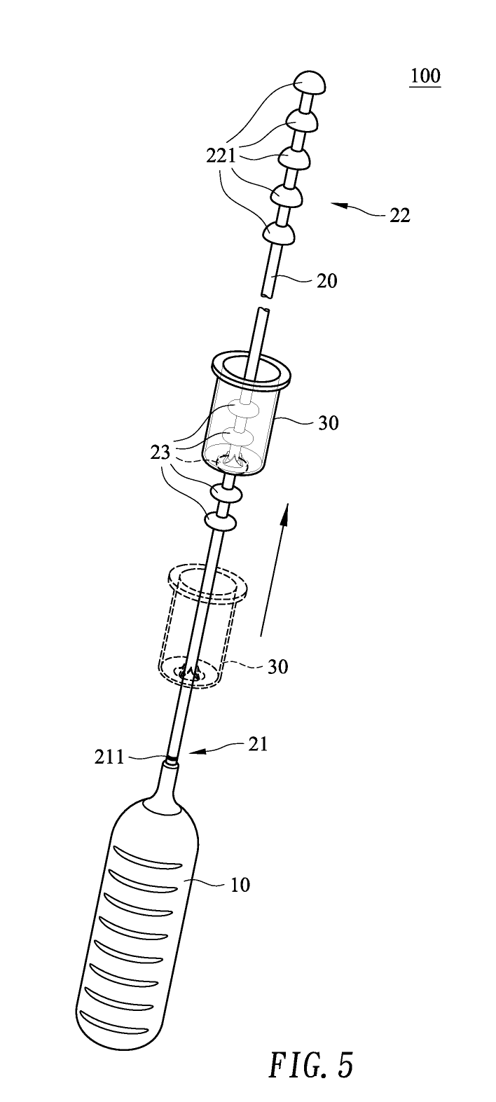

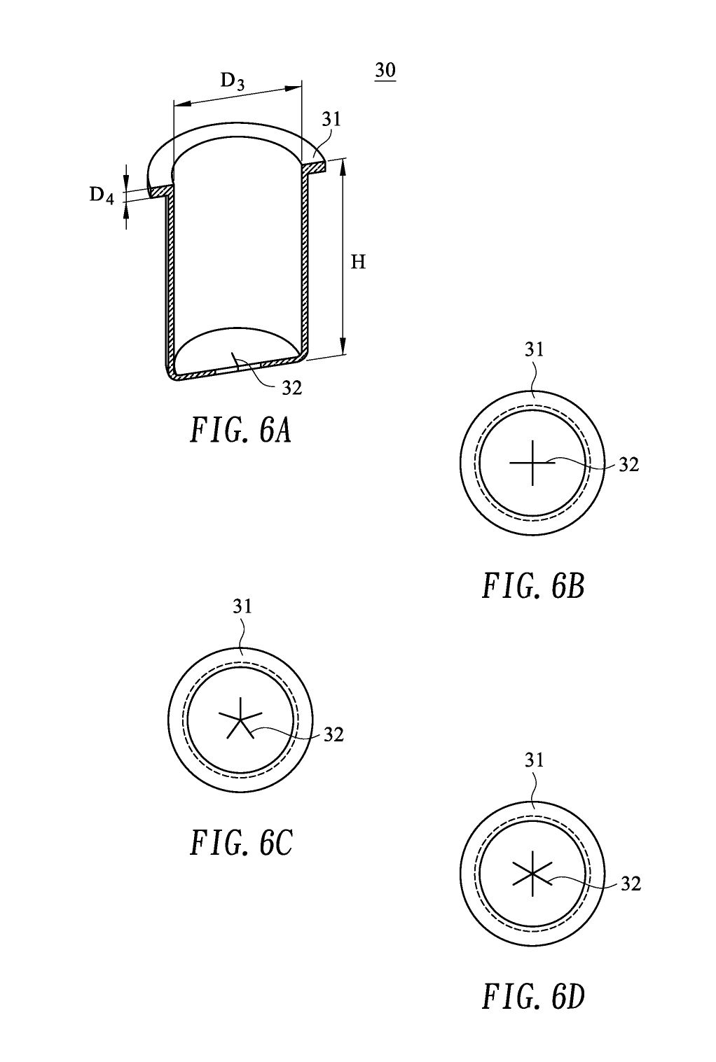

Referring to FIG. 5, the collection cup 30 is slidably connected between the first end portion 21 of the curetting bar 20 and the mushroom-shaped curetting plates 221. The collection cup 30 may be a transparent collection cup so that the operator of the endometrial curette 100 can easily observe through the bottom of the cup, whether a sufficient amount of endometrial tissues has been collected or not. As it is required to place the collection cup 30 against the opening of the cervix during the specimen collecting process, the collection cup 30 must have proper dimensions in order to pass through the vagina without blocking the operator's field of view. For example, referring to FIG. 6A, the bottom of the collection cup 30 has an inner diameter D₃ of 1.1 cm, the height H of the collection cup 30 is 2 cm, and the cup opening of the collection cup 30 is provided with a rim 31, whose thickness D₄ is 0.2 cm.

Referring to FIG. 6A to FIG. 6D in conjunction with FIG. 5, the bottom of the collection cup 30 is centrally provided with a cut 32, such as a cross-shaped cut with four slits converging at a point (see FIG. 6B), a star-shaped cut with five slits converging at a point (see FIG. 6C), a star-shaped cut with six slits converging at a point (see FIG. 6D), or a star-shaped cut with eight slits converging at a point (not shown). The curetting bar 20 can pass through the cut 32, allowing the collection cup 30 to slide between the first end portion 21 of the curetting bar 20 and the lowermost mushroom-shaped curetting plate 221.

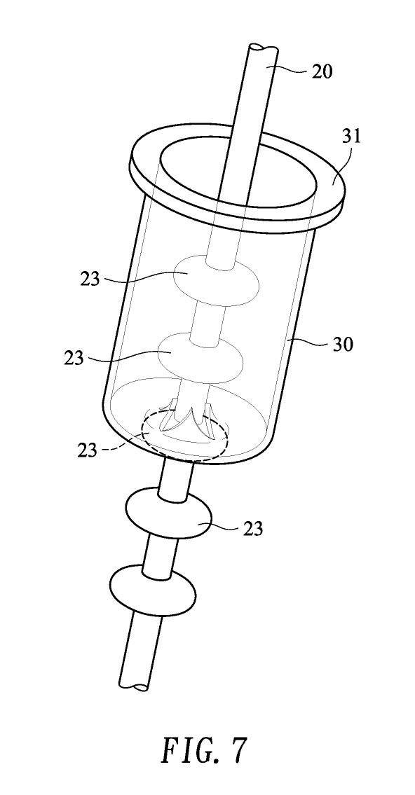

If the bottom of the collection cup 30 were provided instead with an aperture through which the curetting bar 20 can pass to enable installation of the collection cup 30 to the curetting bar 20, endometrial tissues fallen into the collection cup 30 may leak through the aperture. Hence, the optimal design is to provide the cut 32 at the center of the bottom of the collection cup 30. Referring also to FIG. 7, when the curetting bar 20 passes through the cut 32, the portion of the bottom of the collection cup 30 that is adjacent to the cut 32 is turned upward. This upwardly turned portion can help receive the fallen curetted endometrial tissues and prevent the fallen endometrial tissues from coming out of the bottom of the collection cup 30.

Referring to FIG. 3A, FIG. 4, and FIG. 7, in order to keep the collection cup 30 from sliding arbitrarily along the curetting bar 20, a plurality of anti-slide projections 23 are formed between the first end portion 21 of the curetting bar 20 and the lowermost mushroom-shaped curetting plate 221. The collection cup 30 can selectively engage with and be stopped at any of the anti-slide projections 23, and by doing so, the position of the collection cup 30 is adjusted and fixed. The interval of the anti-slide projections 23 may be 0.5 cm, allowing the user to select the appropriate anti-slide projection 23 for engaging with and stopping the collection cup 30.

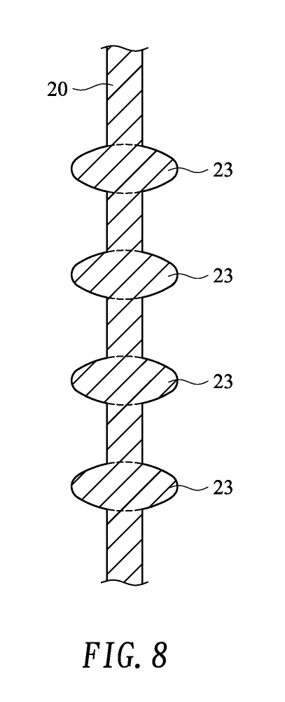

Reference is now made to FIG. 8. The anti-slide projections 23 must be small enough to pass through the collection cup 30 but large enough to engage with and stop the collection cup 30. In consideration of this, each anti-slide projection 23 must correspond in size approximately to the cut 32 of the collection cup 30. Further, as installation of the collection cup 30 entails mounting the collection cup 30 around the curetting bar 20 in a downward direction starting from the top end of the curetting bar 20, the size of the cut 32 of the collection cup 30 preferably exceeds the greatest width of the mushroom-shaped curetting plates 221. Hence, the anti-slide projections 23 may be similar in size to the mushroom-shaped curetting plates 221, i.e., extending 0.05 cm to 0.1 cm from the bar body of the curetting bar 20. More particularly, the anti-slide projections 23 may extend 0.05 cm or 0.1 cm from the bar body of the curetting bar 20, depending on the overall dimensions of the endometrial curette 100.

Assume both the mushroom-shaped curetting plates 221 and the anti-slide projections 23 extend 0.05 cm from the bar body of the curetting bar 20, and the cross-sectional diameter D₁ of the bar body of the curetting bar 20 is 0.2 cm. The greatest width of the mushroom-shaped curetting plates 221, therefore, is 0.3 cm. In that case, the cut 32 of the collection cup 30 may be 0.3 cm or 0.4 cm wide, provided that the collection cup 30 can be mounted around the curetting bar 20. Alternatively, assume both the mushroom-shaped curetting plates 221 and the anti-slide projections 23 extend 0.1 cm from the bar body of the curetting bar 20, and the cross-sectional diameter D₁ of the bar body of the curetting bar 20 is 0.2 cm. Consequently, the greatest width of the mushroom-shaped curetting plates 221 is 0.4 cm, and the cut 32 of the collection cup 30 may be 0.4 cm wide.

In order to take endometrial tissue specimens from inside the uterus by way of the vagina and the cervix, the curetting bar 20 and the collection cup 30 must be made of a plastic material which is medically safe, resilient, freely flexible, and hence adaptable to the internal shape and contour of the uterine cavity. In addition, the length by which the curetting bar 20 is to extend into the uterus must be controlled to ensure patients' comfort, and the greatest cross-sectional width of the curetting bar 20, the mushroom-shaped curetting plates 221, and the anti-slide projections 23 is preferably controlled not to exceed 0.4 cm. The length of the endometrial curette 100 from the bottom of the handle 10 to the top end of the uppermost mushroom-shaped curetting plate 221 may be 24 cm.

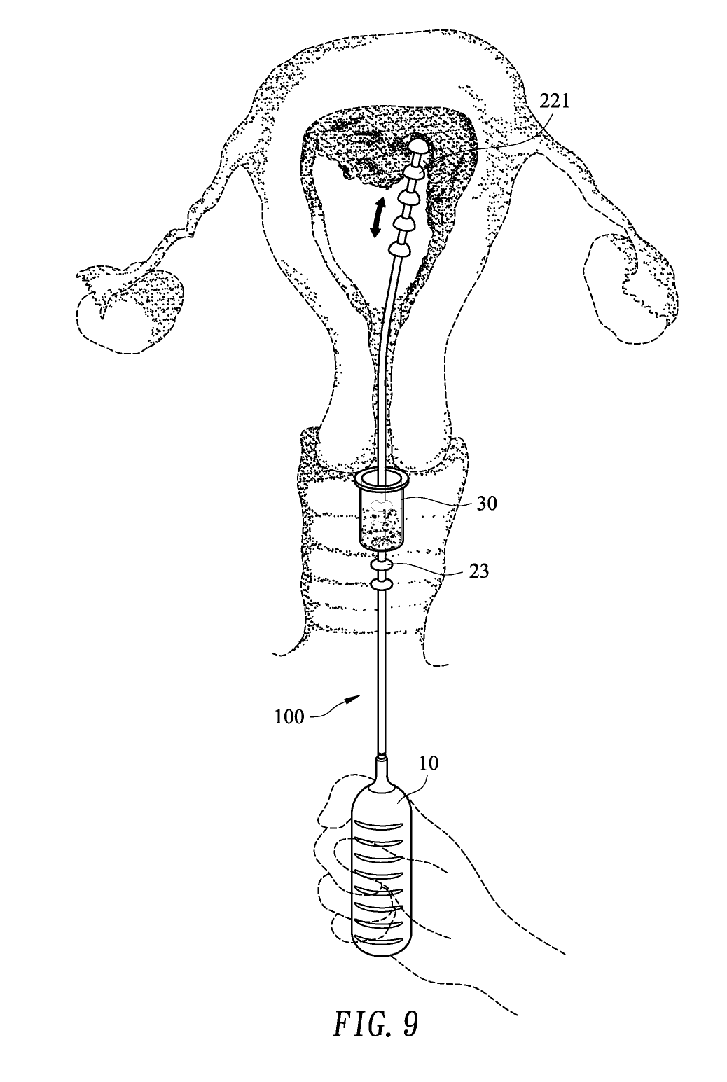

The endometrial curette 100 in the present embodiment is used in the following manner. Referring to FIG. 9, the handle 10 is held with one hand, and the collection cup 30 is stopped and fixed immediately above the handle 10 during curettage.

While the user moves the endometrial curette 100 up and down in the uterus, the mushroom-shaped curetting plates 221 remove endometrial tissues by scraping in all directions. The removed endometrial tissues, either falling freely or drawn out by the endometrial curette 100, can be found at the opening of the cervix. Once the curetting process is completed, the endometrial curette 100 is pulled out of the cervix while the collection cup 30 is moved upward. The collection cup 30 is eventually fixed at, and kept from sliding downward by, an appropriate anti-slide projection 23 and pressed tightly against the opening of the cervix in order to collect all the fallen tissues for examination.

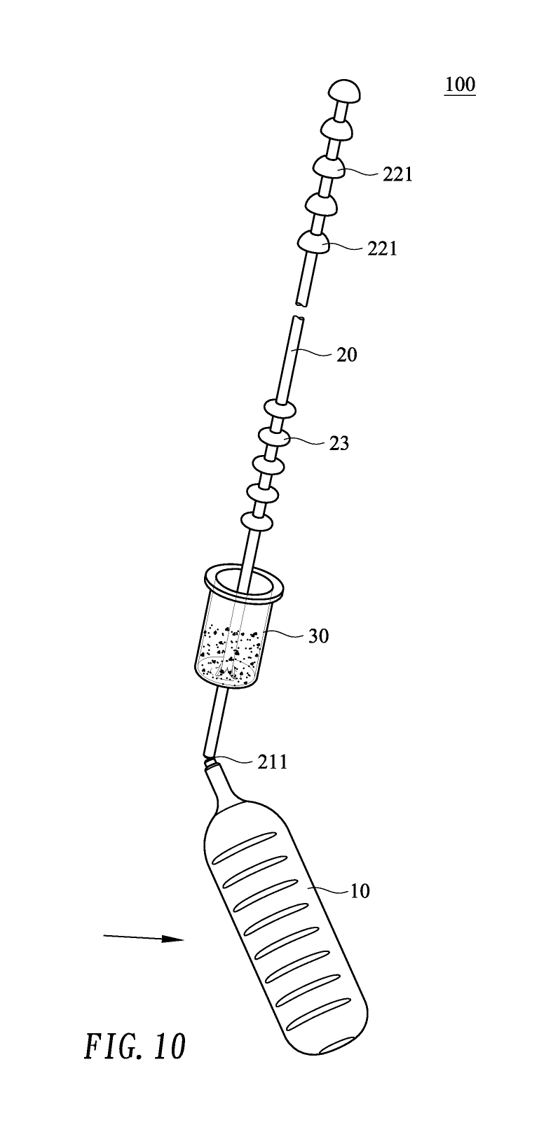

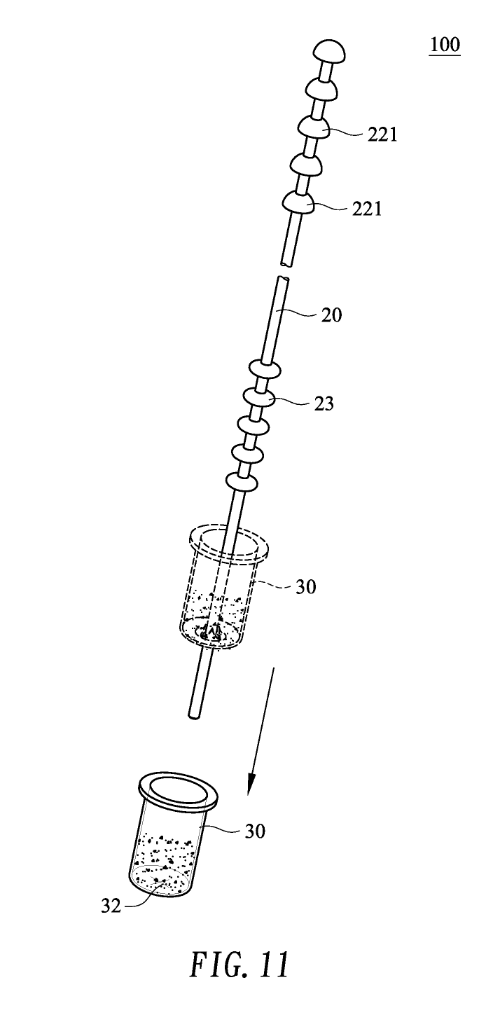

Referring to FIG. 10 and FIG. 11, after a sufficient amount of endometrial tissues is from all dimensions collected, the curetting bar 20 is broken off at the notch 211 and separated from the handle 10. Then, the collection cup 30 is pulled downward along and thereby removed from the curetting bar 20. The portion of the bottom of the collection cup 30 that is adjacent to the cut 32 now resumes its original flat shape to prevent the: collected endometrial tissues from falling out, and the endometrial tissues in the collection cup 30 are ready for pathological examination.

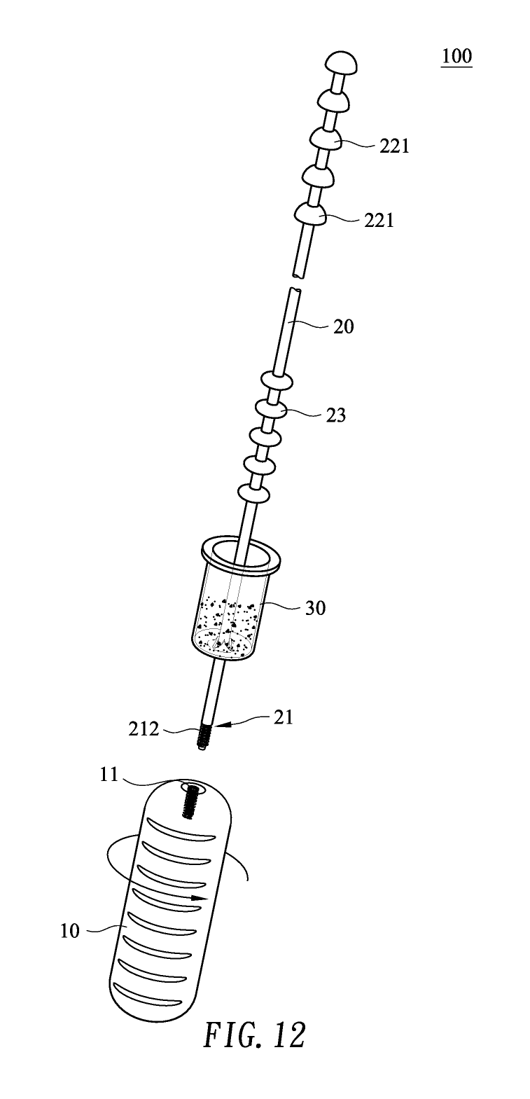

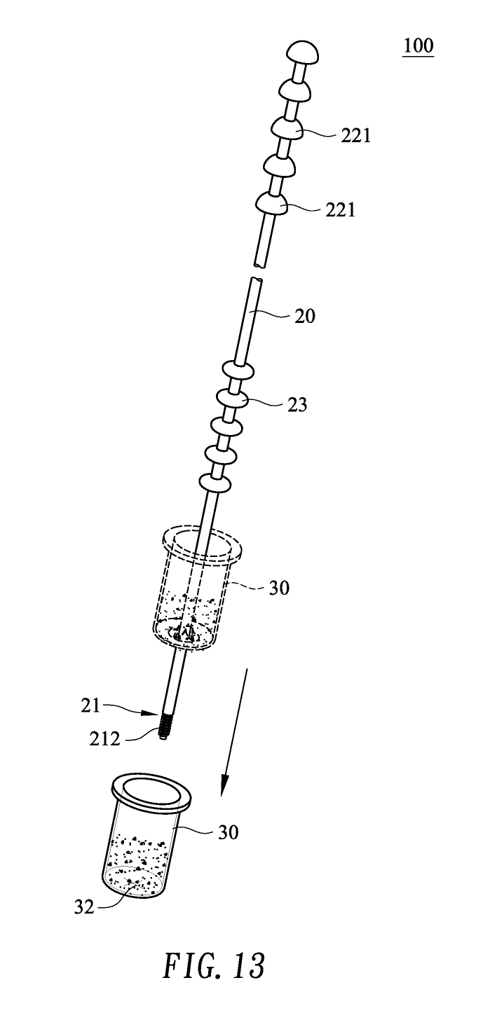

Referring to FIG. 12 and FIG. 13, the curetting bar 20 also can be separated from the handle 10 by unscrewing the screw threads 212 and the threaded hole 11. Before the collection cup 30 needs to be pulled downward, the handle 10 is rotated until the handle 10 is separated from the curetting bar 20. Then, the collection cup 30 can be pulled downward along and thereby removed from the curetting bar 20. Thus, the handle 10 can be reused and connected to another curetting bar 20.

As the diameter of the endometrial curette 100 is merely under 0.4 cm, one who is to receive endometrial curettage by the curette 100 need not undergo cervical dilatation or anesthesia. In addition, now that the discomfort caused by the specimen collecting process is within a tolerable range, specimens can be rapidly taken in an outpatient setting, which is beneficial to the promotion of endometrial cancer screening.

The embodiments described above are intended only to demonstrate the technical concept and features of the present invention so as to enable a person skilled in the art to understand and implement the contents disclosed herein. It is understood that the disclosed embodiments are not to limit the scope of the present invention. Therefore, all equivalent changes or modifications based on the concept of the present invention should be encompassed by the appended claims.

What is claimed is:

1. A step-shaped endometrial curette, comprising:
a handle;
a curetting bar having a first end portion and a second end portion, wherein the first end portion is connected to the handle, and the second end portion has a distal end formed with a plurality of mushroom-shaped curetting plates; and
a collection cup slidably connected between the first end portion and the mushroom-shaped curetting plates.

2. The step-shaped endometrial curette of claim 1, wherein the collection cup has a bottom centrally provided with a cut, and the curetting bar passes through the cut such that the collection cup is slidably connected between the first end portion and a lowermost said mushroom-shaped curetting plate.

3. The step-shaped endometrial curette of claim 2, wherein the cut comprises a plurality of slits converging at a point.

4. The step-shaped endometrial curette of claim 2, further comprising a plurality of anti-slide projections formed between the first end portion and the lowermost mushroom-shaped curetting plate in order for the collection cup to selectively engage with and be stopped at any said anti-slide projection.

5. The step-shaped endometrial curette of claim 4, wherein the curetting bar has a bar body with a cross-sectional diameter of 0.2 cm, the anti-slide projections extend 0.05 cm to 0.1 cm from the bar body of the curetting bar, and the anti-slide projections are arranged at an interval of 0.5 cm.

6. The step-shaped endometrial curette of claim 4, further comprising a notch formed where the first end portion is connected to the handle, such that the curetting bar can be broken off at the notch.

7. The step-shaped endometrial curette of claim 6, wherein the curetting bar has a bar body with a cross-sectional diameter of 0.2 cm, the anti-slide projections extend 0.05 cm to 0.1 cm from the bar body of the curetting bar, and the anti-slide projections are arranged at an interval of 0.5 cm.

8. The step-shaped endometrial curette of claim 1, wherein the first end portion forms with screw threads and the handle forms with a threaded hole so that the handle and the curetting bar is connected by screwing the screw threads into the threaded hole.

9. The step-shaped endometrial curette of claim 8, wherein the handle is reusable.

10. The step-shaped endometrial curette of claim 1, wherein the collection cup is a transparent collection cup.

11. The step-shaped endometrial curette of claim 1, further comprising a notch formed where the first end portion is connected to the handle, such that the curetting bar can be broken off at the notch.

12. The step-shaped endometrial curette of claim 1, wherein each of the curetting bar and the collection cup is made of a medically safe plastic material.

13. The step-shaped endometrial curette of claim 1, wherein the mushroom-shaped curetting plates are perpendicular to a bar body of the curetting bar and are formed at the distal end of the second end portion at an equal interval.

14. The step-shaped endometrial curette of claim 1, wherein there are five said mushroom-shaped curetting plates arranged at an equal interval of 0.5 cm.

15. The step-shaped endometrial curette of claim 1, wherein each said mushroom-shaped curetting plate has a thickness of 0.2 cm and extends 0.05 cm to 0.1 cm from a bar body of the curetting bar.

16. The step-shaped endometrial curette of claim 1, wherein the collection cup has a bottom with an inner diameter of 1.1 cm, has a height of 2 cm, and has a cup opening formed with a rim, the rim having a thickness of 0.2 cm.
